(19) United States
(12) Patent Application Publication
(10) Pub. No.: US 2009/0292147 A1
(43) Pub. Date: Nov. 26, 2009

(54) PROCESS FOR THE CONTINUOUS PRODUCTION OF POLYETHER ALCOHOLS

(75) Inventors: Thomas Ostrowski, Mannheim (DE); Johannes Adam, Dresden (DE); Dirk Meckelnburg, Ludwigshafen (DE); Ulrike Mahn, Mannheim (DE); Renata Jovanovic, Ludwigshafen (DE); Achim Loeffler, Speyer (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, LUDWIGSHAFEN (DE)

(21) Appl. No.: 12/303,477

(22) PCT Filed: Jun. 15, 2007
(86) PCT No.: PCT/EP2007/055934

§ 371 Date: Dec. 4, 2008

(30) Foreign Application Priority Data
Jun. 23, 2006 (EP) 06115946.3

(51) Int. Cl. C07C 43/10

(52) U.S. Cl. 568/620

(57) ABSTRACT

The invention relates to a process for the continuous preparation of polyether alcohols by reacting H-functional initiators with alkylene oxides using basic catalysts, wherein at least one initiator is metered with at least one alkylene oxide continuously into a back-mixing reactor and the reaction product is removed continuously from the back-mixing reactor.

The invention relates to a continuous process for the preparation of polyether alcohols.

Polyether alcohols are produced in large amounts and are widely used. They are generally produced by an addition reaction of alkylene oxides with H-functional initiators. The addition reaction is usually effected in the presence of catalysts, in particular basic compounds, such as amines or alkali metal hydroxides, or of multimetal cyanide compounds, also referred to as DMC catalysts.

The main field of use of the polyether alcohols is the production of polyurethanes. Depending on the requirements with regard to the properties of the polyurethanes, the polyether alcohols may differ greatly in their functionality, the molecular weight and the initiators used.

At present, polyether alcohols are predominantly produced by the batch or semibatch procedure. The DMC catalysts are an exception. Since these catalysts promote the growth of short chains over longer chains, polyether alcohols can also be prepared by a continuous process. Here, initiator and alkylene oxides are metered continuously into a continuous reactor, for example a continuous stirred kettle or a tubular reactor, and the finished product is removed continuously. Such processes are described, for example, in WO 98/03571 or in DD 204 735. However, these processes are limited to DMC catalysts. However, only polyether alcohols as used for the preparation of flexible polyurethane foams can be prepared with the use of liquid initiators by the DMC procedure. The addition reaction of alkylene oxides with solid initiators or with aromatic amines, as used as initiators for the preparation of polyether alcohols for use in rigid polyurethane foams, is not possible with DMC catalysts.

It would be desirable to find a process for the preparation of rigid-foam polyether alcohols which is improved compared with the semibatch process customary at present. The relatively large reactors required in this procedure owing to the long setup and emptying times decisively determine the capital costs of the production plants. Furthermore, valves, pumps and motors are subjected to considerable stress owing to the changing loads. Precisely in the preparation of polyols on the basis of sugar, a strong motor, gears and stirrer axles are moreover necessary in order to be able to mix the sugar slurry thoroughly with the alkylene oxides. The semibatch procedure can moreover result in variations in the product quality from batch to batch.

The semibatch procedure also has disadvantages during the alkoxylation. In general, the sugar is mixed with a co-initiator, such as glycerol, diethylene glycol, triethanolamine, dipropylene glycol or water to form a slurry. The alkylene oxide is then metered in. Particularly at the beginning of the process, however, the propylene oxide is virtually insoluble in the sugar slurry. High pressures therefore initially occur in the reactor so that in certain circumstances the metering of the alkylene oxide has to be stopped. Furthermore, different degrees of alkoxylation are observed.

The conversion of this procedure to a more cost-efficient continuous procedure is not directly possible. Since the alkali-catalyzed propoxylation is a living polymerization, the direct conversion of the semibatch technology, as is possible, for example, in the case of DMC catalysis and is described in U.S. Pat. No. 5,698,012, is not possible. Rather, broad molecular weight distributions are observed in this case.

WO 00136514, WO 00136088 and WO 00136513 describe tubular reactors which can also be used for the preparation of rigid-foam polyether alcohols. In order to achieve complete conversion here, the tubular reactors must be designed very long.

Although the use of tubular flow reactors would lead to narrow molecular weight distributions, the slurry comprising sugar and co-initiators can be pumped with difficulty and, if at all, only by very expensive procedures through the pipelines. The problems of non-miscibility of alkylene oxide phase and sugar phase and consequently the low reaction rates owing to low concentration also persist. Furthermore, numerous metering points are necessary in order to be able to operate the reactor safely and to monitor the reaction temperature.

It was therefore the object to provide an improved process for the continuous preparation of polyether alcohols, in particular those for the preparation of rigid polyurethane foams, which leads to products having a narrow molecular weight distribution, can be operated simply and safely and also permits the use of solid initiators.

The object could surprisingly be achieved if the reaction of the initiator with the alkylene oxide is effected in a back-mixing reactor, in particular a continuous stirred kettle (CSTR).

The invention accordingly relates to a process for the continuous preparation of polyether alcohols by reacting H-functional initiators with alkylene oxides using basic catalysts, wherein at least one initiator is metered with at least one alkylene oxide continuously into a back-mixing reactor and the reaction product is removed continuously from the back-mixing reactor.

Preferably, at least one initiator is at least tetrafunctional.

The term “tetrafunctional” means that the compound has at least 4 reactive hydrogen atoms.

The at least tetrafunctional compounds preferably used as initiators are selected in particular from the compounds customarily used for the preparation of polyether alcohols. These are preferably aliphatic amines, in particular ethylenediamine, and aromatic amines, in particular toluenediamine (TDA) and mixtures of isomers of diphenylmethane diisocyanate and its higher homologs (MDA), mixtures of aromatic and aliphatic amines or solid OH-functional compounds, such as pentaerythritol, carbohydrates, preferably starch, cellulose and particularly preferably sugar, in particular sorbitol, mannitol, glucose, fructose and sucrose. The use of melamine and its H-functional derivatives is also possible.

Since some of the at least tetrafunctional initiators are solid at the reaction temperatures, and, particularly in the case of the sugars and here in particular of sucrose, also cannot be melted without decomposition, particular precautions should be taken for the continuous metering of these substances.

Thus, it is true that it is possible in principle to feed these compounds as solids to the reactor, for example via a tube. However, particularly in the case of sucrose, this can lead to agglomerations and blockages in the tubes. Furthermore, it is possible to dissolve the solid initiator in a solvent, such as water, or to suspend it in compounds which are liquid at the processing temperature and to feed it to the reactor in this form. The feeding can also be effected via pumps or, particularly in the case of highly viscous suspensions, via screw conveyers, for example eccentric screw pumps, screw pumps and other positive pumps.

The compounds which are liquid at the processing temperature may be low molecular weight compounds, in particular difunctional or trifunctional compounds having active hydrogen atoms, in particular alcohols, such as ethylene glycol, propylene glycol or glycerol. In one embodiment of the process according to the invention, these are an intermediate or the end product of the process. This can be worked up particularly by the removal of water or the catalyst, but may also be the crude product. It is also possible to use difunctional to tetrafunctional polyetherols having molecular weights of from 200 to 600 g/mol as co-initiators. These polyetherols may be catalyst-free or may comprise a catalyst. It is also possible here to use a catalyst other than that used in the actual continuous process. It is therefore also conceivable to introduce a further catalyst into the reaction by the use of the alkaline additional polyetherol.

In one embodiment of the process according to the invention, with the use of solid initiators, a reactor in which the solid initiator is liquefied by an addition reaction of a small amount of alkylene oxide and this precursor is metered continuously into the continuous back-mixing reactor is connected upstream of the continuous back-mixing reactor. Said upstream reactor may be a continuous or a batchwise reactor. With the use of a batchwise reactor in this preliminary stage, the precursor is usually temporarily stored in storage tanks and fed from there continuously to the back-mixing reactor. With the use of a continuous reactor in this preliminary stage, the precursor can be metered directly to the back-mixing reactor or likewise temporarily stored in storage tanks and fed from there continuously to the back-mixing reactor. The required amount of alkylene oxide which is subjected to an addition reaction with the solid initiator, in particular the sucrose, is variable. The aim is to subject alkylene oxide to an addition reaction in an amount such that the solid initiator is liquefied.

It is also possible to use a part of the finished product, before or after the removal of the catalyst, as a co-initiator. This product recycling results in the solid initiator being dispersed in a larger mass of liquid initiator.

This preliminary stage can be particularly advantageously used if the initiator is initially insoluble in the reaction mixture present in the continuous back-mixing reactor and becomes soluble only after the addition reaction of alkylene oxide, as, for example, in the case of sucrose.

The continuous back-mixing reactor may be a continuous stirred kettle reactor, a jet loop reactor having an internal heat exchanger, as described, for example, in DE 19854637 or in DE 10008630, or a jet loop reactor having an external heat exchanger, as described, for example, in EP 419 419. It is particularly preferably a continuous stirred kettle.

The continuous back-mixing reactor, in particular the stirred kettle, may be designed to be individual or in the form of a cascade. The reactor size depends on the required residence time and can be determined in the customary manner by the person skilled in the art. It should preferably be chosen at least so that the desired degree of alkoxylation at which the added solid initiator dissolves in the reaction medium is reached.

The heat removal during the exothermic alkoxylation can be effected via an external heat exchanger. In order to prevent residual sugar from being transported into the further process steps, the product can be removed from the external cooling circulation after passing a cross-flow filtration unit. The sugar-containing stream is recycled to the stirred kettle while the sugar-containing product stream is passed into the further process step.

In one embodiment of the process according to the invention, the prepared polyether alcohol is removed from the back-mixing reactor.

In another embodiment of the process according to the invention, a further continuous reactor is connected to the continuous back-mixing reactor. Said further continuous reactor may likewise be a back-mixing reactor but is preferably a tubular reactor. This is also referred to below as postreactor.

In one embodiment, this postreactor serves for the complete conversion of alkylene oxide still present in the discharge from the continuous back-mixing reactor. In this embodiment, no alkylene oxide is metered into the postreactor. If required, further catalysts may be added to the reaction mixture before or during the reaction in the postreactor. This may be the same catalyst as in the continuous back-mixing reactor or another catalyst.

It is also possible to design the downstream reactor so that the solid initiator is completely converted and no content of solid initiator remains in the product. Excess alkylene oxide present can then be stripped off.

In a further embodiment of the process according to the invention, the molecular weight of the product from the continuous back-mixing reactor is further increased in the postreactor. For this purpose, if appropriate further alkylene oxide and, if appropriate, further initiator, in particular liquid initiator, which may comprise alcohols, amines or the alkoxylates thereof, are metered into the postreactor. In the case of a tubular reactor, the metering can be effected directly at the entrance of the postreactor and/or at at least one metering point in the course of the tubular reactor.

The preferably used tubular reactor may be of different designs. Thus, it is possible to use an empty tube. Preferably, the tube can be designed with internals, for example with packings, static mixers with or without internal heat exchanger surfaces and/or internals which lead to the formation of plug flow, for example commercially available SMX, SMR types from Sulzer or as a helical tube reactor. The heat removal can be effected via the jacket or an internal cooling coil in the tubular reactor or by intermediate cooling with the aid of inserted heat exchangers or heat exchangers provided between tube sections. As described, metering points for initiator, alkylene oxides and catalysts can be installed at one or more points of the tubular reactor. It is also possible to operate the reactor without additional metering points. In this embodiment, the alkylene oxide required for the reaction in the further reactor is added to the reaction mixture before the entry into this reactor.

The residence times in the further reactors depend either on the requirement to obtain a concentration of free propylene oxide below 5%, preferably below 1%, after the reactor. Alternatively, the postreactor can be designed so that the residual content of solid initiator is minimized, preferably to a residual content of less than 0.5% by weight. The flow rates in the tubular reactors should be chosen so that radial mixing in the reaction medium is achieved which results in only small radial temperatures and concentration gradients, if any at all. This can be achieved via a turbulent flow profile, internals, such as packings or static mixers, or a coiled tube.

As in the continuous back-mixing reactor, the reaction temperatures should be chosen so that firstly a high reaction rate can be achieved and secondly damage to the product is avoided.

The concentrations of free alkylene oxides at the feed points should be chosen so that the heat removal after the reaction is ensured.

A conceivable embodiment would be a tubular reactor which is divided into sections separated from one another, so-called compartments, by closed plates, which compartments are connected to one another by external pipelines in which the discharge of the reactor from the first step of the reaction flows through the compartments in succession. No alkylene oxide is metered into this reactor.

The at least tetrafunctional initiators can be used alone or in combination with other H-functional compounds, so-called co-initiators. Possible co-initiators are alcohols and amines having 1-6 functional groups which can react with alkylene oxides. In particular, these are difunctional or trifunctional alcohols, aminoalcohols or amines. These compounds are liquid at least at room temperature and should improve the pumpability and flowability of the reaction mixture and establish the functionality of the prepared polyether alcohol. Examples of these are glycerol, ethylene glycol, propylene glycol, ethanolamine, diethanolamine, triethanolamine, diethylene glycol, dipropylene glycol and water and the lower alkoxylates thereof (molecular weight 200-600 g/mol). In the postreactor, where the metering of initiator is effected, preferably those having a functionality of not more than 3 are used.

As described, basic compounds are used as catalysts. These are usually tertiary amines and/or hydroxides of alkali metals and alkaline earth metals. Examples of amine catalysts are trimethylamine, tributylamine, triethylamine, dimethylethanolamine and dimethylcyclohexylamine. Examples of the hydroxides are potassium hydroxide, sodium hydroxide, strontium hydroxide and calcium hydroxide.

Said catalysts can be used individually or as a mixture with one another. It is possible to use the same or different catalysts in the continuous back-mixing reactor and in the further reactor.

In a particular embodiment of the process according to the invention, amine catalysts are used in the continuous back-mixing reactor and metal hydroxides in the further reactor.

The catalyst concentration may be from 0.01 to 10%, based on the total mass of the polyol. If readily volatile amines, such as TMA or TEA are used, it is also possible to separate them off from the end product by means of stripping or distillation and to reuse them. If required, the catalysts, in particular metal hydroxides, can be removed after leaving the tubular reactor. Here, it is possible to employ crystallization processes with the use of mineral acids, such as phosphoric acid, adsorption processes with the use of acidic adsorbents, such as Ambosol, and/or ion exchange processes. It is moreover possible to carry out only a neutralization by means of organic acids, such as acetic acid, lactic acid, citric acid or 2-ethylhexanoic acid, or mineral acids instead of complete or partial removal of the catalyst.

The process according to the invention is carried out as usual at temperatures of from 50 to 180° C. The pressure during the reaction in the CSTR is 1-40 barg, and the pressure in the tubular reactor should be chosen so that the alkylene oxides remain liquid for the most part but as far as possible completely. Under certain circumstances, for example with the use of vertical tubular reactors, however, a gas phase could be present.

The concentration of free alkylene oxide should be 1-40% in all process steps.

After the complete addition reaction of the alkylene oxides, the polyether alcohol is worked up in the customary manner.

Thus, it may be necessary to remove residues of alkylene oxides by stripping in columns, bubble columns or evaporators, in particular thin-film evaporators or falling-film evaporators. At the same time, any volatile catalysts present, in particular amines, can be removed.

Alkylene oxides and/or volatile catalysts removed at the end of the reaction cascade or between two reactors within the cascade can either be discarded or can be used again as starting materials. On the one hand, it is possible to feed in the substances as liquids after condensation. On the other hand, the gaseous reaction components can also be introduced into the reaction mixture with the use of a suitable absorber, for example an absorber column. The introduction of the recycled alkylene oxide and/or catalyst can be effected both at the beginning of the reactor cascade and between two reactors. The initiator mixture or parts thereof can be used as absorbents.

The removal of the catalysts based on alkali metal or alkaline earth metal, which is necessary depending on the application, can be effected by classical methods, for example crystallization, ion exchange or adsorption. In this case, products which have an alkalinity of <200 ppm are strived for. Such processes should preferably be continuously operated.

Stabilization of the products by antioxidants can be effected if this is required, for example for reasons relating to the application or the shelf-life.

By means of the process according to the invention, it is possible to prepare polyether alcohols in a simple and effective manner by a continuous process. The polyether alcohols prepared by the process according to the invention are distinguished by a narrow molecular weight distribution and low color numbers. Furthermore, owing to the continuous procedure, a constant product quality is achieved. The contents of unconverted initiator molecules, in particular of the solid initiators, is low, as a rule below 0.1% by weight.

The polyether alcohols prepared by the process according to the invention preferably have a molecular weight in the range of 200-2000 g/mol, in particular 200-1000 g/mol.

The invention is to be explained in more detail with reference to the following examples.

### EXAMPLE 1

### COMPARISON

The apparatus consisted of a stirred kettle having an anchor stirrer and a heating/ cooling jacket which was thermostated by means of an oil thermostat. The temperature regulation and monitoring were effected via an internal thermocouple. The reactor was hydraulically filled and was operated at a constant pressure of 30 barg. A pressure control valve which allowed the reactor content continuously into the product receiver under reduced pressure (about 40 mbara) was positioned at the reactor exit. The reaction temperature was 110° C. The sugar/glycerol mixture was introduced into the reactor via a slurry metering pump. Catalyst and propylene oxide were metered via separate HPLC pumps.

45.4 g/h of sugar as a mixture with 16.6 g/h of glycerol were metered. The catalyst KOH was metered as a 50% strength solution (0.4 g/h). The metering rate of the propylene oxide was 137.6 g/h.

The reaction product was analyzed with respect to OH number, residual sugar content and foamability. The residual sugar content was determined by silylating the product and then analyzing it by gas chromatography. The product which was obtained in steady-state operation (after about 5 residence times) was analyzed. The product had a high residual oxide content of 10% (sampling before degassing and gas chromatographic analysis). The content of free sugar was on average 3%. The OH number of the product was 502 mg KOH/g.

### EXAMPLE 2

### COMPARISON

The same metering apparatuses as in example 1 were used. Instead of the stirred kettle reactor, a DN25 tubular reactor having Fluitec CX static mixers was used. The reactor consisted of 20 elements of 260 mm length. In each case at the beginning of the first 15 elements, propylene oxide was metered at an intermediate flange. The temperature in the tubular reactor was measured by means of thermocouples having ceramic insulation in the intermediate flanges and removed via a heating/cooling jacket. Here too, oil thermostats having an external water cooler were used. The last 5 elements served only as a zone for completing the reaction. A pressure control valve which allowed the reactor content continuously into the product receiver under reduced pressure (about 40 mbara) was positioned at the reactor exit. The temperature was 120° C. The sugar/glycerol mixture was introduced into the reactor via a slurry metering pump. Catalyst and propylene oxide were metered via separate HPLC pumps.

During the experiment, blockages in the region of the first two elements by crystalline sugar occurred after short operating times (<0.5 h). The sugar additionally had a dark color (was caramelized). Temperatures up to 168° C. were measured at the temperature measuring points. The temperature peaks occured a short time before the blockages.

It was not possible to take any product sample from the steady-state operation since the total residence time in the tube was longer than the maximum duration of operation achieved. Polyetherol released from the tube behind the blockages likewise had a dark color.

### EXAMPLE 3

The apparatus consisted of a stirred kettle (1.4 l volume) having a 3-stage crossbeam stirrer and a heating/cooling jacket which was thermostated by means of an oil thermostat. The temperature regulation (120° C.) and monitoring were effected by means of an internal thermocouple and an oil thermostat with a water cooler. The reactor was hydraulically filled and was operated at a constant pressure of 30 barg. A DN25 tubular reactor having Fluitec CX static mixers was installed downstream of the stirred kettle. The reactor consisted of 6 elements of 260 mm length. In each case at the beginning of the first 4 elements, propylene oxide was metered at an intermediate flange. The temperature in the tubular reactor was measured by means of thermocouples having ceramic insulation in the intermediate flanges and was removed via a heating/cooling jacket. Here too, oil thermostats having an external water cooler were used. The last 2 elements served only as a zone for completing the reaction. A pressure control valve which allowed the reactor content continuously into the product receiver under reduced pressure (about 40 mbara) was positioned at the reactor exit. The reaction temperature was 110° C. The sugar/glycerol mixture was introduced into the reactor via a slurry metering pump. Catalyst and propylene oxide were metered via separate HPLC pumps.

45.4 g/h of sugar as a mixture with 16.6 g/h of glycerol were metered. The catalyst KOH was metered as 50% strength solution (0.4 g/h). The metering rate of the propylene oxide was:

| Metering location | Metering rate |
|---|---|
| CSTR | 50% |
| Tube 1 | 12.5% |
| Tube 2 | 12.5% |
| Tube 3 | 12.5% |
| Tube 4 | 12.5% |

In the CSTR, a propylene oxide content of about 5% was established; after the tubular reactor, the propylene oxide content was about 1% (sampling and gas chromatographic analysis).

### EXAMPLE 4

The apparatus consisted of a stirred kettle (1.4 l volume) having a 3-stage crossbeam stirrer and a heating/cooling jacket which was thermostated by means of an oil thermostat. The temperature regulation (120° C.) and monitoring were effected by means of an internal thermocouple and an oil thermostat with a water cooler. The reactor was hydraulically filled and was operated at a constant pressure of 30 barg. A DN25 tubular reactor having Fluitec CX static mixers was installed downstream of the stirred kettle. The reactor consisted of 6 elements of 260 mm length. The temperature in the tubular reactor was measured by means of thermocouples having ceramic insulation in the intermediate flanges and was removed via a heating/cooling jacket. Here too, oil thermostats having an external water cooler were used. The tubular reactor served only as a zone for completing the reaction. A pressure control valve which allowed the reactor content continuously into the product receiver under reduced pressure (about 40 mbara) was positioned at the reactor exit. The reaction temperature was 110° C. The sugar/glycerol mixture was introduced into the reactor via a slurry metering pump. Catalyst and propylene oxide were metered via separate HPLC pumps.

45.4 g/h of sugar as a mixture with 16.6 g/h of glycerol were metered. The catalyst KOH was metered as a 50% strength solution (0.4 g/h). The metering rate of the propylene oxide was 137.6 g/h.

The concentration of the propylene oxide in the CSTR was about 20%; no significant concentration of propylene oxide was found after the tubular reactor (sampling and gas chromatographic analysis).

1-22. (canceled)

23. A process for the continuous preparation of polyether alcohols by reacting H-functional initiators with alkylene oxides using basic catalysts, wherein at least one initiator is metered with at least one alkylene oxide continuously into a stirred kettle reactor and the reaction product is removed continuously from the stirred kettle reactor.

24. The process according to claim 23, wherein the continuously operated stirred kettle reactor is arranged as a stirred kettle cascade.

25. The process according to claim 23, wherein a further continuous reactor is present downstream of the continuously operated stirred kettle reactor.

26. The process according to claim 23, wherein the further continuous reactor is a reactor having plug flow.

27. The process according to claim 23, wherein the further continuous reactor is a tubular reactor.

28. The process according to claim 23, wherein further alkylene oxide is metered into the further continuous reactor.

29. The process according to claim 23, wherein no further alkylene oxide is metered into the further continuous reactor.

30. The process according to claim 23, wherein at least one initiator is at least tetrafunctional.

31. The process according to claim 23, wherein the at least tetrafunctional initiator is an aromatic amine.

32. The process according to claim 23, wherein the at least tetrafunctional initiator is a carbohydrate which is solid at room temperature.

33. The process according to claim 23, wherein the at least tetrafunctional initiator is a sugar.

34. The process according to claim 23, wherein the solid initiator is liquefied by reaction with alkylene oxide prior to the metering into the continuously operated stirred kettle reactor.

35. The process according to claim 23, wherein the catalysts used are amines.

36. The process according to claim 23, wherein the catalysts used are hydroxides of alkali metals and/or alkaline earth metals.

37. The process according to claim 23, wherein the catalysts used are mixtures of amines and hydroxides of alkali metals and/or alkaline earth metals.

38. The process according to claim 23, wherein the amine catalyst used is stripped off after the continuously operated back-mixing reactor.

39. The process according to claim 23, wherein the amine catalyst stripped off is recycled to the continuously operated stirred kettle reactor.

40. The process according to claim 23, wherein the amine catalyst stripped off is not recycled to the continuously operated stirred kettle reactor.

41. The process according to claim 40, wherein the catalyst stripped off is absorbed by the reaction medium via a suitable apparatus.

42. The process according to claim 24, wherein the unreacted alkylene oxide is stripped off after the reaction cascade or between two reactors and is recycled to a preceding reactor.

43. The process according to claim 42, wherein the alkylene oxide stripped off is absorbed by the reaction medium via a suitable apparatus.

44. The process according to claim 25, wherein the residual content of solid initiator after the further reactor is below 0.5% by weight.

45. The process according to claim 32, wherein the residual content of solid initiator after the further reactor is below 0.5% by weight.
